(19) United States
(12) Patent Application Publication
(10) Pub. No.: US 2018/0028806 A1
(43) Pub. Date: Feb. 1, 2018

(54) ORTHOSIS

(71) Applicant: OTTO BOCK HEALTHCARE GMBH, Duderstadt (DE)

(72) Inventors: Alexander HARDT, Asslar (DE); Juergen KAUS, Bad Wildungen (DE)

(73) Assignee: OTTO BOCK HEALTHCARE GMBH, Duderstadt (DE)

(21) Appl. No.: 15/662,115

(22) Filed: Jul. 27, 2017

(30) Foreign Application Priority Data
Jul. 29, 2016 (DE) 102016114074.0

(51) Int. Cl. A61N 1/36; A61F 5/01; A61F 5/058

(52) U.S. Cl. CPC A61N 1/36003; A61F 5/0585; A61F 5/0123; A61F 5/0127; A61F 2005/0167

(57) ABSTRACT

An orthosis having a first splint, which has a fastening device to fix the first splint to a first body part, an joint connected to a second splint, which in a donned/attached state of the orthosis bears on a second body part, and at least one electric stimulation device, which is adjustably fastened to the orthosis.

### TECHNICAL FIELD

The present disclosure relates to an orthosis having a first splint, which has a fastening device for fixing the first splint to a first body part, a second splint, which in the donned/attached state of the orthosis bears on a second body part, and a joint connecting the first and second splints together.

### BACKGROUND

Orthoses are orthopedic devices which are worn on the body, for example, on a limb, a body part, or the torso of a wearer, and are fixed to the respective limb or to the body part with fastening elements and fastening devices such as straps, bands, or clips. The fastening devices may be closed using buckles or hook-and-loop fasteners, for example, to fix the fastening devices to the respective limb or to the body part in a manner that is individually adapted to the orthosis wearer. Orthoses facilitate, stabilize, support, and/or restrict the movement of the respective limb or joints, and may likewise be employed and configured for correcting defective positions, for guiding the body parts, for correctly aligning or maintaining a specific orientation, and for protecting joints and limbs or body parts.

In the region of the lower extremities, orthoses may be configured as a knee-ankle-foot orthosis (KAFO) or as an ankle-foot orthosis (AFO), for example, and may minimize the effects of a weakening or of a failure of the foot lifting musculature, for example. A spring-loaded joint may be provided in the region of the natural ankle joint and pretension a foot plate in relation to a lower leg splint in order to carry out dorsal flexion in the swing phase such that a swing through of the foot is possible without the toes or the tip of the foot scraping the ground. Instead of a spring-loaded joint, a spring may also connect the foot plate to the lower leg splint.

An orthosis for correcting a defective leg position, having a bearing bracket, which engages below a foot of a person and establishes contact with a running surface, e.g., a sole or a floor, is known from EP 2 563 300 B1. The orthosis has an upwardly protruding splint arrangement that is disposed beside the leg so as to be lateral thereto and is connectable to the lower leg of the person with a fastening device, wherein an angle piece, which under stress is rigid, is configured on the transition from the bearing bracket to the splint arrangement. The splint arrangement includes a rotary joint, which is disposed so as to be approximately level in height with the natural ankle joint, and is subdivided into a bearing bracket that bears laterally on the foot and a splint that bears laterally on the lower leg. The splint is configured as a spring element. A correcting force, which acts laterally on the lower leg, is exerted using a torque that results from an adjusted pretension of the spring element relative to the lower leg. As long as no pretension of the spring element relative to the lower leg is adjusted, such an AFO serves as a guide splint when plantar flexion and dorsal flexion of the foot is possible, e.g., a movement of the foot may be performed in the sagittal plane. However, pivoting in the medial or lateral direction is prevented or at least impeded. A resistance to a pronation or supination of the foot is provided such that a movement in the frontal plane is prevented or impeded.

WO 03/003953 A1 relates to an adjustable flexion and extension stop of an orthosis, which is configured as a ground reaction orthosis. Sensors are provided to detect the step of a patient. A functional electric stimulation is switched on or off using feedback from the sensors.

U.S. Pat. No. 5,121,747 A relates to a hybrid orthosis for facilitating the movement of a paraplegic. The orthosis has an joint lock, sensors, and electrodes for stimulation of an extension and flexion movement. In order for the stimulation signal to be triggered, the position of the ground reaction force vector is determined mechanically with a tape which bears on a patella cushion. An electrode for stimulating the quadriceps is activated if the tension of the tape exceeds a predetermined value. The electrode is adhesively bonded directly to the skin.

### SUMMARY

One aspect of the present disclosure relates to an orthosis which is easy to put on, provides adequate guidance, and simultaneously enables an effective electric stimulation. Advantageous design embodiments and refinements of the present disclosure are disclosed in the description and the figures.

An orthosis according to the present disclosure includes a first splint which has a fastening device for fixing the first splint to a first body part, a second splint, which in the donned/attached state of the orthosis bears on a second body part, a joint connecting the first and second splints together, and at least one electric stimulation device fastened to the orthosis. The stimulation point which is formed by the electrodes is established and defined in an unequivocal and reproducible manner by the electric stimulation device being fastened to the orthosis. Concurrently with putting on the orthosis, it is possible for the electric stimulation device and the electrodes that are required for the electric stimulation to be disposed in a reproducible manner exactly on the desired position of the body part and to be affixed there concurrently with the orthosis. By combining an orthosis with a functional electric stimulation and by fastening the electric stimulation device, in particular the electrodes for stimulating the musculature still remaining, to the orthosis, the mutual guidance of the body parts with the splint construction is ensured, and the movement functions such as lifting of the foot, stabilization of the knee, or flexion and extension are provided using the functional electric stimulation. Apart from the stabilization by the splint arrangement, a stabilization of the movement and joints is additionally performed with the muscle stimulation, and an improved functionality is achieved by an improved execution of movements.

The orthoses according to the present disclosure may be advantageously employed in the case of patients having neurological damage and in the case of orthopedic secondary damage such as arthrosis. Neurological patients often suffer imbalances, for example, in the upper subtalar joint, leading to amplified supination or reduced pronation. This creates the risk of an inversion trauma when walking, which may be limited by the orthosis according to the present disclosure. To this end, the unstable subtalar joint is stabilized in the supination/pronation direction by the electric stimulation, but is released in the planar/dorsal direction so as to enable safe and natural walking.

Since a precise control of the stimulation may be difficult and many disturbance variables may arise, the orthosis serves as a stabilization instrument, as a guidance instrument, and/or as a movement restriction device, and has the task of counteracting the inadequacies in the stimulation. Should the stimulation system fail or not function as desired, a correct mutual coordination of the body parts or limbs continues to be ensured by the orthosis.

The stimulation may make it possible for neuronal or muscular issues to be alleviated, for example, in the case of a so-called drop foot following a stroke or a slipped disc. The functional electric stimulation thus alleviates the primary indication, while the orthosis prevents secondary damage or compensates for the latter in that instabilities are balanced and damage to or on the joint is compensated for or prevented. Primary damage may be defined as that damage that has a comparatively large effect.

Using dissimilar stimulation of muscles may make it possible for the muscular balance to be influenced and for the mutual coordination of the body parts to be influenced solely or in conjunction with the orthosis. For example, medial arthrosis may be addressed by a stimulation of the lateral musculature. Lateral arthrosis may be treated, corrected or balanced by a stimulation of the medial musculature. It is thus possible for defective mutual positions of body parts to be compensated for or for symptoms in the case of arthrosis to be treated by therapy solely or in conjunction with the respective design embodiment of the orthosis.

While the orthosis stabilizes and guides, the locationally fixed electrodes or electrode arrangements may be capable of being placed in a reversible and repeatable manner in the predetermined locations of the body parts. Potential consequential damage of stimulation alone, in particular any hyperextension or overstretching of ligaments, is avoided by the guidance of the orthosis.

The orthosis may have fastening devices on the first splint as well as on the second splint. It may be possible for a fastening device to be disposed only on a first splint, for example, a lower leg splint, while the second splint is configured as a foot part or a foot stirrup such that fixing of the second splint is performed by an arrangement in a shoe. It is likewise also possible for both splints to have a fastening device such that in each splint is fixed to a separate body part and a natural joint provides a connection of the splints to one another. Apart from the design embodiment as an AFO, an orthosis having one joint and two splints may also be configured as a pure knee orthosis in which the orthosis joint is located level in height with the natural knee joint, and the splints extend from the joint in the proximal and distal direction along the thigh and the lower leg.

In one embodiment of the present disclosure, a third splint is connected to one of the other splints via the joint, and the third splint in the donned/attached state of the orthosis bears on a further third body part. In an embodiment in which the orthosis is designed as a KAFO, the first splint may be fixed to a lower leg, the second splint represents a foot part or a foot stirrup, and the third splint is configured as a thigh splint and, with the second joint, e.g., the orthosis knee joint, is connected to the first splint. Further splints may be interconnected in an articulated manner. A full-leg orthosis having a hip socket, a so-called HKAFO (hip-knee-ankle-foot orthosis), may include such a third splint.

In order for a full-leg orthosis or KAFO to be able to be securely fixed to a leg, the second fastening device for fixing a further splint, apart from the first splint, to another body part is preferably provided on the third splint. It may be possible for three or more fastening devices to be provided on the orthosis. For example, a plurality of fastening devices may be disposed on one splint. A fastening device may likewise be present above the foot part or the foot stirrup in order for the foot part or foot stirrup to be fixed to the foot below the ankle.

In one refinement of the present disclosure, the electric stimulation device, in particular having the electric stimulation electrodes, is fixed to a splint and/or to a fastening device. A plurality of electric stimulation devices may be present, for example, in order for various groups of muscles to be stimulated or activated, for example. By disposing and fixing the electric stimulation device to one splint it is possible for the position of the electric stimulation device to be fixed relative to the muscle to be stimulated. The electric stimulation device may be disposed within a coverage region of the splint such that the electric stimulation device is affixed directly to the splint. However, this arrangement may restrict the possibilities for positioning the electrodes.

There is furthermore the possibility for the electric stimulation device to be fixed to holding elements or the like on the splint, for example, in the form of clamps, clasps, bands, or straps. The fastening devices and/or holding elements may be displaceably and/or torsionally mounted on the respective splint. It may be possible for muscles or activation points that are more remote from the splint to be reached. When the fastening device and/or the holding element is displaceably mounted on the splint, there is the possibility for the holding device to be affixed to the splint in a desired position.

One embodiment of the present disclosure includes an electric stimulation device, in particular the electrodes, which is displaceably mounted, e.g., longitudinally displaceable, on the splint or splints. A fixed assignment to the surface of the body part is maintained by virtue of the fastening to the splints. An adhesive may be used to limit slippage on the skin surface. The adhesive may be disposed on the inner side of the electrodes, on the inner side of the fastening device, and/or on the inner side of the holding elements. A fixed assignment of the electric stimulation device in relation to the skin surface may also be ensured by the fastening device per se, for example, by a contact pressure that is applied when putting on the orthosis or by a surface design embodiment. Relative movements resulting from alignment errors between the joint axes and the axes of the natural joints may be equalized by a repositioning capability of the electrode relative to the splint in the donned/attached state. However, when the orthosis is being put on, the electrodes are located at the assigned spot, enabling a correct assignment of the electrodes to the desired stimulation points on the skin surface.

An electric stimulation device is preferably employed in combination with a fastening device for fixing one of the splints to a body part, such that the electric stimulation device, in particular the stimulation electrode or the stimulation electrodes, is/are disposed within the fastening device. The fastening device preferably encompasses the body part in a circumferential manner, at least in a partially circumferential manner, and may be configured as a clasp, a strap, a clip, or the like. The fastening device may be configured as a spring or articulated in order for a modification of the circumference to be able to be readily effected. The fastening device is preferably actuatable with one hand, for example, in that a flexible clasp is placed in a partially circumferential manner about the body part and is subsequently closed in a fully circumferential manner by, for example, a hook-and-loop fastener, a toggle fastener, an elastic strap, or a screw fastener, in order for the fastening device to be fixed to the body part so as to be adapted to the body part.

The electric stimulation device has at least one electrode, e.g., a surface electrode, which is disposed on the inner side of the splint, the inner side of the holding element, or the inner side of a fastening device, such that the electrode bears directly on the skin surface of the respective body part to be stimulated in the region of the musculature. The electric stimulation device may also have at least one electrode arrangement, wherein electrode arrangements have electrodes in pairs. Electrodes, as referenced herein, are analogous electrode arrangements. The electric stimulation device is coupled to a control unit which activates the electrodes depending on, for example, the spatial position, movement data of the respective splint or limb or of the body part, for example, the acceleration, load, angular position, or contact sensors, so as to stimulate the respective musculature in response to a stimulation signal. The sensors may also be disposed on the untreated contralateral leg, so as to initiate control on the basis of the movement data of the healthy leg there.

The splints are preferably freely pivotable about a pivot axis that is formed by the joint. The pivot axis is preferably oriented in the direction of the natural joint axis of the respectively assigned natural joint, so as to provide an unimpeded movement of the joint in preferably only one plane. The other movement planes may be restricted or blocked such that precise mutual guidance of the limbs or body parts is provided by the orthosis. It may also be possible for the respective orthosis to be configured as a corrective orthosis and to act on the mutual orientation of the limbs or body parts with a pretension. The joint per se preferably has no resistance to movement such that free pivoting about the preferably stationary pivot axis is enabled. In some embodiments, no spring elements for pretensioning one splint in relation to the other splint in a specific direction are provided within the joint.

In one embodiment of the present disclosure, the second splint is configured as a foot part that engages below (e.g., along a bottom surface of) a foot and the first splint is configured as a lower leg splint. The lower leg splint is preferably disposed laterally on the limb or on the body part, such as on the lower leg. Alternatively thereto, it is possible for the lower leg splint to be disposed in a medial manner and to bear on the inner side of the lower leg. In principle, it is also possible for two mutually opposite lower leg splints to be mounted to a single lower leg, which in each case are guided in an joint that is level in height with the natural ankle joint, so as to provide improved guidance and to preclude tilting of the foot relative to the lower leg in the frontal plane. The joint or the joints may be disposed so as to be level in height with the natural ankle joint, which may enable ready pivotability without impeding the mutual pivoting movement of the limb or the body parts.

One embodiment provides that the third splint is configured as a thigh splint and the second knee joint is located so as to be level in height with the natural knee joint, so as to also enable an unimpeded pivoting movement of the lower leg relative to the thigh here. A medial-lateral arrangement of the thigh splint on both sides of the thigh is also possible in such a design, such that lower leg and thigh are in each case enclosed medially and laterally by two splints.

The splints provide guidance to the limb and body parts in a medial-lateral direction, limit any mutual disorientation of the limb and the body parts, and enable a guided movement within those pivot planes that are defined by the splints about the respective joint axis.

The splints in terms of their mutual inclination are adjustable in a reversible manner. This may be performed either by a plastic deformation of the splints, or by a design embodiment of the splints in multiple parts as splint elements, wherein the splint elements are embodied in a curved manner and are disposed so as to be inclined in respective receptacles provided therefor in the end regions of the splint elements. It is possible for the respective inclination of the splints to be adapted to the patient by releasing the splint elements in the receptacles and by corresponding twisting and affixing. A prefabricated orthosis component may thus be readily adapted to the respective user. It is likewise possible for a pretension of the orthosis to be adjusted to the limb or the body parts in the donned/attached state by adjusting the inclination of the splints in a targeted manner relative to the joint device or in a mutually relative manner to the splints, so as to implement a design embodiment of the orthosis as a corrective orthosis.

Markings or previously fixed fastening elements for the electric stimulation devices, in particular for the electrodes, may be disposed on the splint and/or the fastening device or the fastening devices, respectively, so as to readily find again, should a readjustment be performed, the once located position of the electrode or of the electric stimulation device, respectively, on the orthosis once the most suitable position of the electric stimulation device or of the electrode for the respective muscle has in each case been established. These markings may be disposed at regular spacings along the splint, for example, in order to enable a readjustment in terms of height. It may also be possible for the fastening device to be attached to the respective splint so as to be longitudinally displaceable or rotatable. After the orthosis has been put on in the previously adjusted state, a locking mechanism may be released such that a relative movement is possible between the fastening device and the splint, for example, in order to enable a repositioning by virtue of eccentricities in the axes and the fastening devices along the splint or a fastening device.

One embodiment of the present disclosure provides that the joints or splints have stops that provide a restriction of the movement. The stops and thus the maximum movement angles possible are preferably adjustable such that an individual adaption to the patient or to a course of therapy is possible. The stops limit unintentional contraction intensity in the case of a stimulation of the musculature that may lead to damage, or hyperflexion or hyperextension. Moreover, the body parts are mutually guided in one plane with the orthosis such that the mutual coordination of the body parts in this plane is maintained during the movement, independently of whether any electric stimulation is performed and how intensive the electric stimulation is.

### BRIEF DESCRIPTION OF DRAWINGS

Illustrative embodiments of the invention are explained in more detail below with reference to the attached figures, in which:

FIG. 1 shows an AFO in a front view;

FIG. 2 shows a variant of FIG. 1, in a side view;

FIG. 3 shows a further design embodiment of an AFO;

FIG. 4 shows a design embodiment of the orthosis as a KAFO;

FIG. 5 shows a design embodiment of the orthosis as a hip-thigh orthosis;

FIG. 6 shows a variant of FIG. 5, having a flexion stop; and

FIG. 7A shows a motion study of an orthosis engaging across the knee.

FIG. 7B shows a close up view of a portion of the orthosis shown in FIG. 7A.

FIG. 8 illustrates schematically a control device for use with the devices of FIGS. 1-7B.

FIG. 9 is a flow diagram illustrating steps of a method in accordance with the present disclosure.

### DETAILED DESCRIPTION

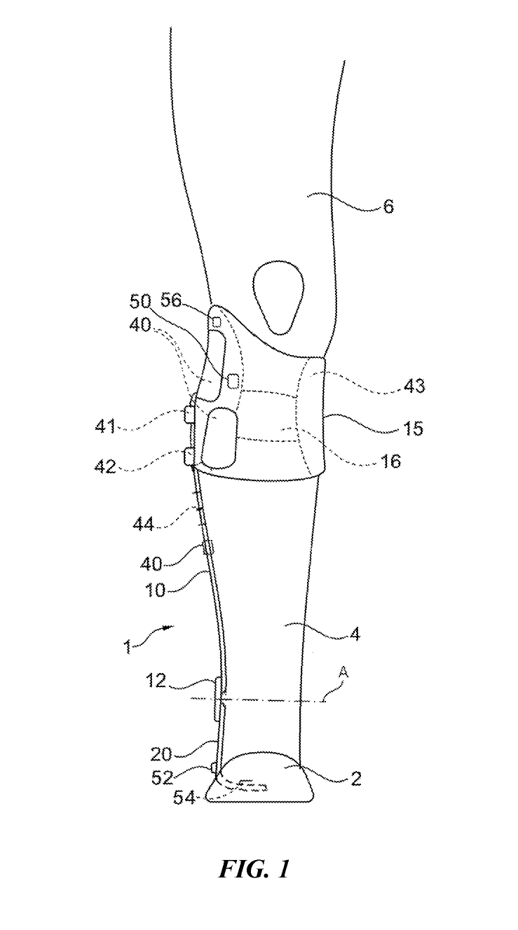

FIG. 1 is a schematic illustration showing an orthosis 1 having a first splint 10 in the form of a lower leg splint, which with a fastening device 15 in the form of a partially encircling band, is fixed to a lower leg 4. The band 15 is fixed to the lower leg 4 in a partially circumferential manner such that the band 15 is opened out and is placed about the lower leg 4, enclosing the lower leg 4 in a frontal as well as medial and lateral manner. A free space is provided on the rear side such that the band 15 or the bracket may be readily opened out. The fastening device 15 is placed completely about the lower leg 4 using a fastener 16, for example, a hook-and-loop fastener. In some embodiments, the band 15 and/or fastener 16 is positioned at least partially below the natural knee joint, such as the rotation axis of the natural knee joint. The band 15 and/or fastener 16 may be positioned in its entirety below the natural knee joint (e.g., the rotation axis of the natural knee joint).

The fastening device 15 is disposed on the first splint 10 so as to be adjustable in height. Affixing devices 41, 42 may be used to adjust the height and optionally also the rotational orientation about the longitudinal axis of the lower leg 4. Affixing devices 41, 42 may be positioned on the transverse extent of the first splint 10, disposed on the first splint 10, or disposed on the fastening device 15. It may be possible with the affixing devices 41, 42 for reversible affixing of the fastening device 15 to the first splint 10. Markings 44 may indicate the adjusted position which is determined by an orthopedic technician, for example, an may be disposed or configured on the first splint 10. The markings 44 may make it possible for the position of the fastening device 15 relative to the first splint 10, once found, to be found again in the case of any erroneous readjustment or of cleaning, or in the case of a replacement of components.

The first splint 10 is coupled to a second splint 20 with the joint 12, so as to be pivotable about a pivot axis A. The second splint 20 is configured as a foot part or a foot stirrup. In the case of a foot stirrup, one component of the foot stirrup extends perpendicularly downward next to the ankle in the direction of the sole of the foot, and the second component of the foot part or foot stirrup extends below the foot and bears on the ground, or at least is connected to a base plate. The pivot axis A may be substantially level in height with the natural ankle joint axis, or substantially level in height with the so-called compromise center of rotation for the ankle joint. Pivoting of the lower leg part or of the first splint 10 relative to the foot part or the second splint 20 is possible about one axis only, whereas any other movement is prevented or at least impeded by the stability of the orthosis 1. The foot portion of the second splint 20 engages below a schematically illustrated foot 2, such as a long a bottom surface of the foot 2.

An electric stimulation device 40 in the form of electrodes, which are disposed on an electrode band 43 on the inner side of the fastening device 15, may be disposed on the orthosis 1. The electric stimulation device 40 or the electrodes 40 are coupled to a control device 50 which on the basis of sensor values, for example, stimulates the respective muscle to be stimulated. This is performed by exerting a current pulse such that the muscle that is close to the electrode 40 is stimulated to contract.

The electrodes 40 may be releasably mounted so as to be displaceable and/or rotatable on the orthosis 1. The orthosis 1 may include predetermined fastening regions, for example, press studs, hook-and-loop fastener regions, or markings, which may be disposed or configured within the fastening device 15, so as to facilitate assigning the electrodes 40 to the respective muscle areas to be stimulated. In the donned/attached state of the orthosis 1 as illustrated, the foot 2 is may be guided relative to the lower leg such that pivoting is possible only about the axis A, that is to say pivoting within a sagittal plane. A lateral movement of the lower leg within the frontal plane, is prevented or limited by the guidance of the orthosis 1. Medial-lateral guiding of the lower leg 4 relative to the foot 2 is thus performed.

As an alternative to fastening the electrodes 40 to the fastening device 15, it is possible for the electrodes 40 to also be mounted directly to the first splint 10, as shown in FIG. 1. This arrangement limits the positioning possibilities in the circumferential direction. Alternatively or additionally to an arrangement on the first splint 10 of the orthosis 1 or the fastening device 15, it is possible for a further mounting of the electric stimulation device 40 or the electrodes 40, respectively, to both of the split 10 and the fastening device 15, or to other features of the orthosis. For example, it is possible to dispose the electrodes 40 in an optimal manner on the lower leg 4, independently of an optimized arrangement of a fastening device 15.

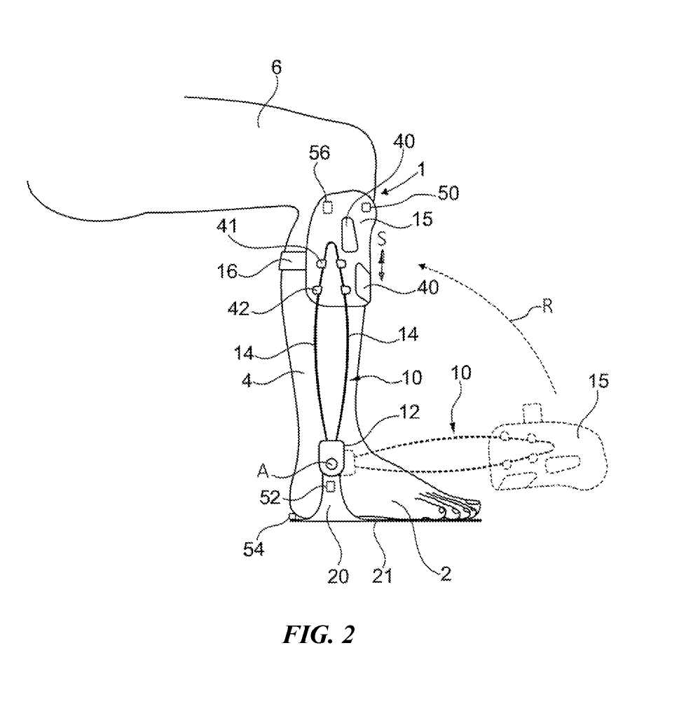

FIG. 2 shows another embodiment of the orthosis 1 according to FIG. 1, in a lateral side view. The second splint 20 has a foot plate 21 and a lateral bracket, which extends from the foot plate 21 laterally in the direction of the ankle. The first splint 10 has two splint elements 14, which are configured so as to be curved and are mounted so as to be capable of torsion and be affix in an joint body, which is pivotable about the pivot axis A. By applying a torsion force and subsequent affixing within the joint body it is possible for an inclined adjustment of the bearing point of the upper end of the first splint 10 on the lower leg to be changed. Accordingly, either a pretension may be applied to the lower leg 4, or else tension-free bearing of the first splint 10 on the lower leg 4 may be effected. The fastening device 15 is configured as a substantially dimensionally stable elastic clasp or clip, which is disposed in a lateral and frontal as well as medial manner about the lower leg 4 in the region of the knee joint. The fastener or closing device 16 (also referred to as a closing element or a rear closing element) in the form of, for example, a hook-and-loop tape enables putting on and closing with one hand such that the fastening device 15, after the orthosis 1 has been put on, always remains in the same position. The electrodes 40 are disposed on the inner side of the fastening device 15, the electrodes 40 by virtue of the assignment and the fixed arrangement within the fastening device 15 having a high positional stability such that these electrodes at all times have the same position once the orthosis 1 has been put on and is being worn.

Affixing devices 41, 42 for the affixing capability of the fastening device 15 on the first splint 10, more specifically on the splint elements 14, are disposed on the fastening device 15. As is indicated by the double arrow S in FIG. 2, the fastening device 15 may be displaced along the longitudinal extent of the first splint 10 when the affixing devices 41, 42 are released. Once the desired position on the splint elements 14 has been reached, the affixing devices 41, 42 are activated and the fastening device 15 is fixed to the first splint 10.

The free pivotability of the first splint 10 about the pivot axis A, which is ideally congruent with the ankle joint axis, is illustrated in FIG. 2. In order to be put on, the foot 2 is placed on the foot plate 21, the ankle is positioned next to the joint 12, and the first splint 10, which is initially folded forward, is pivoted in the direction of the arrow R in the direction towards the lower leg 4. The fastening device 15 is then positioned conjointly with the electrodes 40 at the correct point on the skin surface. The stimulation point for the electrodes 40 is established by putting on the orthosis 1. Alternatively to the arrangement of the electrodes 40 within the clasp, the electrodes 40 may also be disposed on the rear closing element 16, on a separate hook-and-loop element, on a separate clasp, band, or the like. The holding elements for the electrodes 40 are adjustable and affixable in terms of height and position. Triggering pulses for the electrodes 40 may be supplied with, for example, a heel switch, an inertia sensor, or other sensors or combinations of sensors 52, 54, 56. The electric energy for the pulses may be supplied on demand by an energy accumulator which may be disposed in the fastening device 15 or may be affixed in a separate support on the user of the orthosis. The connection between the energy accumulator and the electric stimulation device 40 in this instance is performed with a cable.

Such a hybrid orthosis having a medial-lateral guidance and a functional electric stimulation may also be employed in the case of patients having a highly pronounced paralysis of the musculature for lifting and lowering the foot, in the case of quadriceps paralysis, for example, following a stroke, in the case of multiple sclerosis, in the case of trauma patients, or the like. For the application on a patient having quadriceps paralysis it is necessary for an electric stimulation device 40 to be disposed as least on the thigh 6.

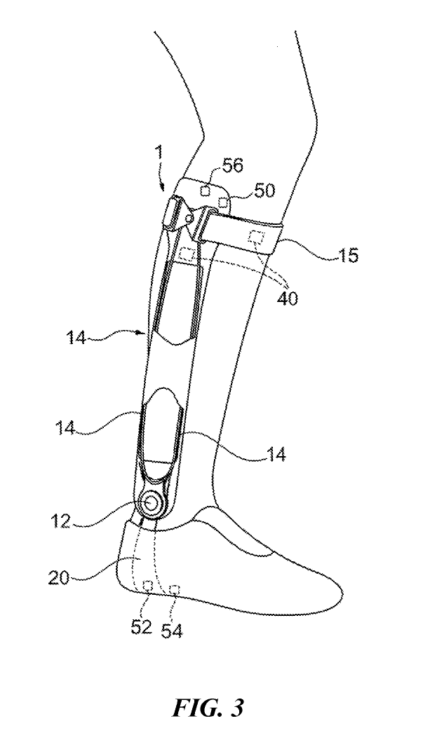

FIG. 3 shows an embodiment of the present disclosure in which the orthosis 1 is fixed to the lower leg 4, not with a clasp which encloses the lower leg in the tibia region, but with a hook-and-loop fastener as the fastening device 15. The electrodes 40 are located on the inner side of the fastening device 15 and/or on the inner side of the cushioning which extends between the splint elements 14 and the surface of the lower leg.

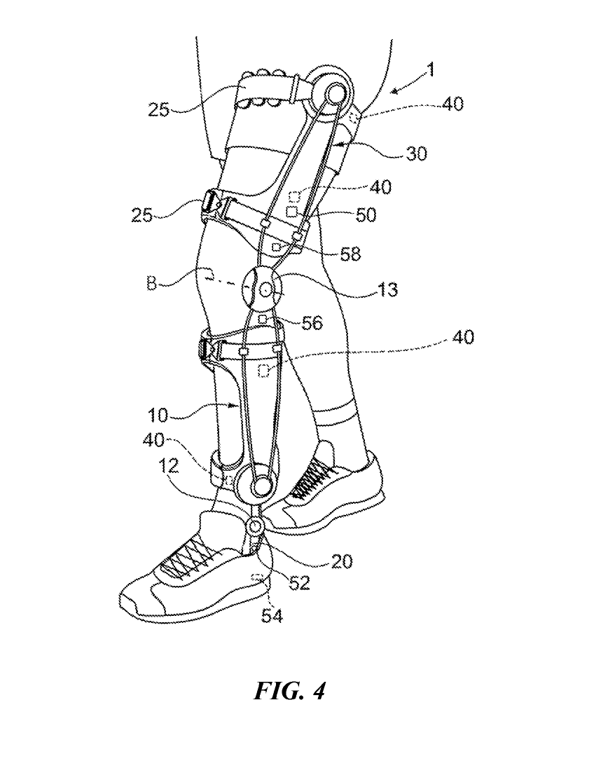

An embodiment of an orthosis 1 as a KAFO is illustrated in FIG. 4. The orthosis includes a first splint 10 as the lower leg splint, a first joint 12 level in height with the ankle, a second splint 20 as the second splint, a third splint 30 having a second joint 13, which is level in height with the natural knee joint. The third splint 30 is secured to the thigh with a fastening device 25. In order for the adaptation to the thigh contour to be enabled, or in order for a pretension to be effected, in order for a defective knee position or a defective ankle position to be compensated for, electrodes of the electric stimulation device may also be disposed on the thigh splint 30, which is likewise configured having two curved splint elements that are capable of torsion, so as to activate the respective muscle, for example, the quadriceps, in order to effect an extension of the lower leg. The pivot axis B of the second joint 13 corresponds to the axis of the natural knee joint. Control of the movement in the sagittal plane and stabilization counter to a movement in the medial-lateral direction are provided with the full-leg splint or KAFO. The control over the musculature in the sagittal plane, which is activated for effecting a dorsal flexion or a plantar flexion of the foot 2 or an extension or flexion of the lower leg 4, is performed with the electric stimulation device 40 which is controlled with one or more sensors 52, 54, 56, 58 in the form of, for example, a gyroscope, an angle sensor, and/or a contact switch in the heel region.

An improved locational stability of the electrodes 40 is achieved by integrating the electrodes 40 in the orthosis 1. Putting on the hybrid orthosis 1 is facilitated and may readily be performed by the user of the orthosis himself/herself without any specialist personnel, once an initial fitting has been carried out. The orthosis 1 is of modular construction such that an AFO is producible by removing the thigh splint 30 from a KAFO, or a pure knee orthosis is producible by removing the second splint 20, without any constructive effort having to be invested. The position of the electrodes may be adjusted in an optimal manner on account of a sliding suspension or other adjustment of the electrodes on the respective splint. Moreover, the orthosis offers additional safety in the case of a failure of the functional electric stimulation, since no sudden supination of the feet and no dangerous stepping on the edge of the foot may arise.

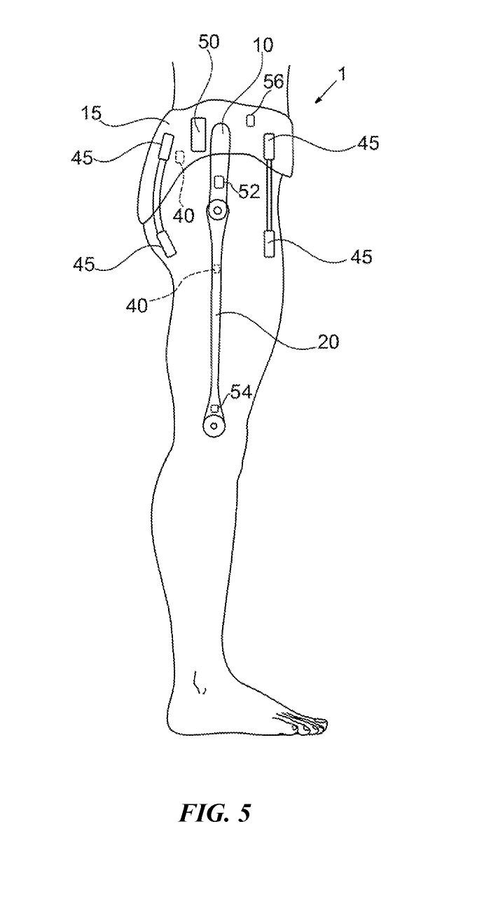

FIG. 5 shows another embodiment of an orthosis 1, having two electrode arrangements 45 having electrode pairs which are positioned on a first fastening device in the region of the hip. The electrodes of the electrode arrangement 45 are disposed in the region of the hip flexor and of the hip extensor and may thus effect a hip flexion and a hip extension on account of a respective stimulation. The electrode arrangements 45 are coupled selectively in a wireless or wired manner to a control device 50. Sensors (e.g., sensors 52, 54, 56), processing devices for sensor signals, and an energy accumulator may be disposed in the control device 50, so as to supply the electrodes of the electrode arrangements 45 with the respective electrical pulses. A first splint 10 is fixed to the fastening device 15 and extends up to the height of the natural hip joint. A second splint 20 is connected in an articulated manner to the first splint 10 with the joint 12 and serves for guiding the thigh relative to the hip. The second splint 20 may be fastened to the thigh with a second fastening device (not illustrated). It may be possible for the lower leg to also be guided with a third splint that is disposed so as to be distal to the second splint 20. It is possible for a flexion or extension of the lower leg relative to the thigh to be initiated or intensified using additional electrodes 40 or electrode arrangements 45 in the region of the thigh musculature, wherein the splints in relation to one another effect mutual guidance of the body parts. The movement ranges, e.g., the angles of flexion or extension, may be adjusted using stops (not illustrated), such as one extension stop for the lower leg relative to the thigh, or for the lower leg splint relative to the thigh splint, respectively, being particularly expedient in order to avoid hyperextension when the lower leg extensor is being stimulated.

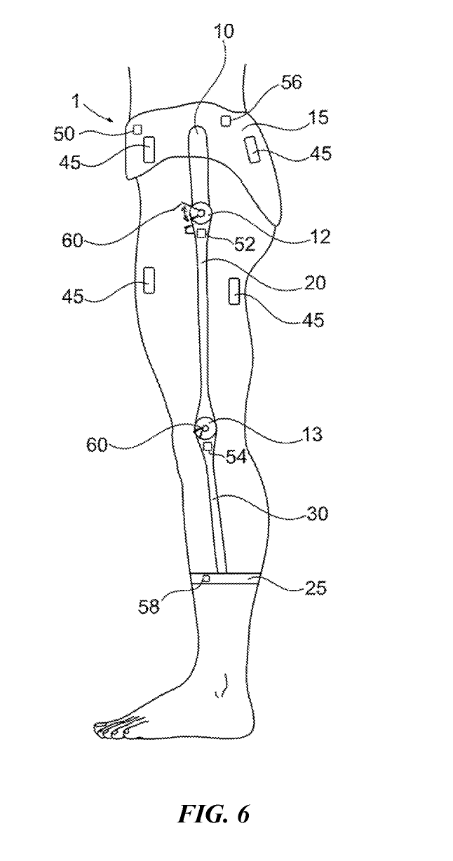

FIG. 6 shows a variation of the orthosis 1 according to FIG. 5, in which a flexion stop 60 for restricting the pivot angle between the first splint 10 and the second splint 20 is disposed. The flexion stop 60 may be configured so as to be adjustable in order for the maximum flexion of the thigh relative to the pelvis to be restricted. An extension stop 60 between the second splint 20 and the second splint 30 (e.g., lower leg splint), the two splints being intercoupled with a further joint 13, is also schematically illustrated in the region of the knee joint. The second splint 30 is affixed to the lower leg with a second fastening device 25.

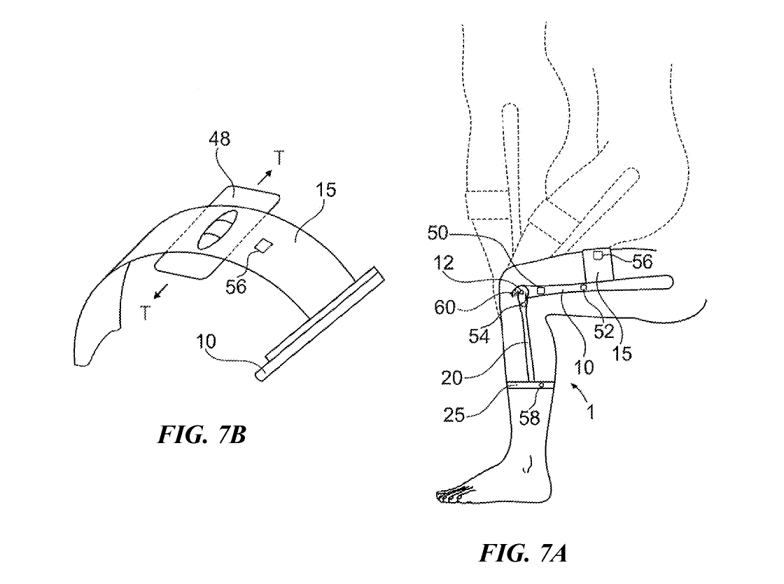

A further variant of the present disclosure in which the orthosis 1 is configured as an orthosis engaging across the knee, having a first splint 10 (e.g., thigh splint) and a second splint 20 (e.g., lower leg splint), is illustrated in FIGS. 7A and 7B. The two splints 10, 20 are interconnected with an joint 12, which is disposed in the region of the natural knee joint axis. Both splints 10, 20 are fixed to the respective body part, e.g., the thigh or the lower leg, with fastening devices 15, 25. FIG. 7A shows various phases of a patient having an orthosis according to the present disclosure standing up. Standing up of the patient is initiated or at least facilitated by activating the quadriceps muscle with the electrodes.

FIG. 7B shows part of the orthosis of FIG. 7A in a detailed view. The first splint 10 is fixed to the thigh by the fastening device 15 with a reusable fastener. The fastening device 15 has an orthosis bracket or an orthosis strap, which may be closed in a circumferential manner such that the orthosis bracket or orthosis strap is capable of being put on about the thigh and is fixable thereto, for example, with straps, buckles, or a hook-and-loop fastener. An electrode holder 48, which is displaceable along the double arrows T and in the respectively adjusted position is affixable to the fastening device 15 or fixable to the thigh strap is disposed on the inner side of the orthosis bracket or of the orthosis strap. The displacement direction of the electrode holder 48 in the exemplary embodiment illustrated runs transversely to the longitudinal extent of the thigh bracket or of the thigh strap, e.g., parallel with the longitudinal extent of the thigh. It may also be possible for a further displacement possibility to be enabled in the longitudinal extent of the thigh strap or of the thigh bracket.

The stop 60 may make it possible for the extension angle of the lower leg, or of the second splint 20, respectively, to be adjusted relative to the thigh or to the thigh splint 10. Medial and lateral splints may be affixed to the thigh so as to achieve improved guidance of the lower leg relative to the thigh. It is likewise possible for any imbalance of the musculature to be balanced or for arthrosis to be counteracted by a dissimilar activation of the medial and/or lateral musculature.

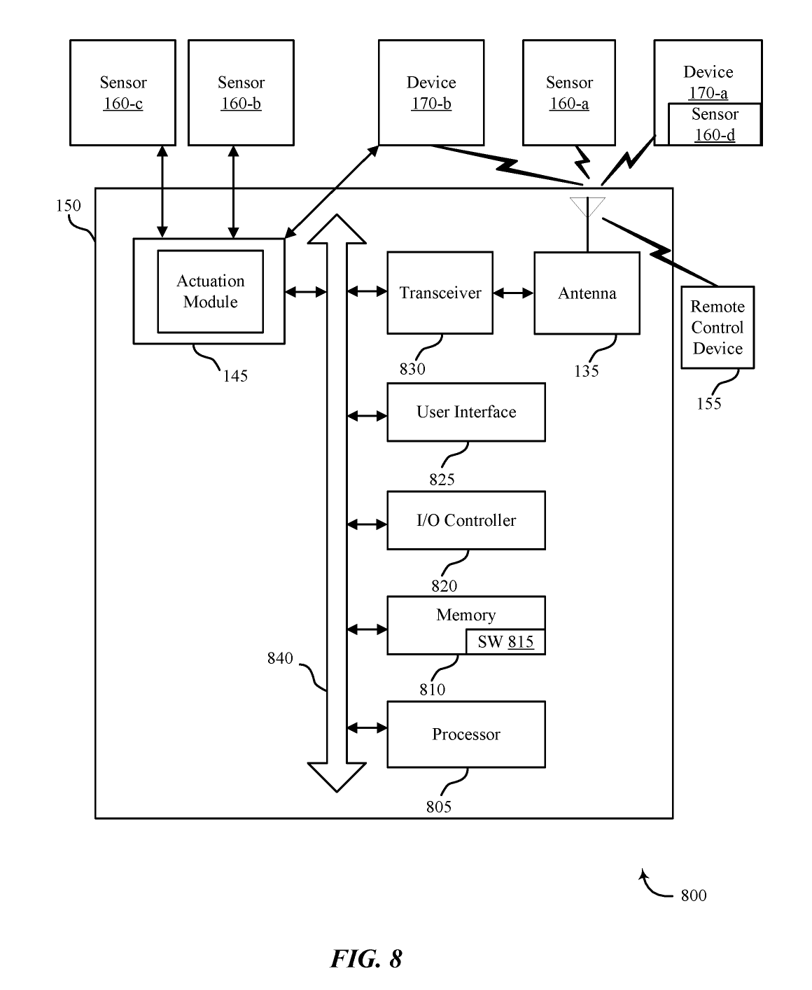

FIG. 8 shows a system 100 for use with the orthosis 1 shown in FIGS. 1-7B. System 100 may include a control device 150. Control device 150 may be equivalent at least in part to the control device 50 described above. Control device 150 may include actuation module 145. The actuation module 145 may provide communications with one or more sensors 160 directly or via other communication components, such as a transceiver 130 and/or antenna 135. The sensors 160 may represent one or more of the sensors 52, 54, 56, 58 described above with reference to FIGS. 1-7B. The actuation module 145 may perform or control various operations associated with, for example, the electronic stimulation device 40 and/or associated electrodes 40, electrode arrangements 45, and other components of the orthosis 1, which are described above with reference to FIGS. 1-7B.

Control device 150 may also include a processor module 105, and memory 110 (including software/firmware code (SW) 115), an input/output controller module 120, a user interface module 125, a transceiver module 130, and one or more antennas 135 each of which may communicate, directly or indirectly, with one another (e.g., via one or more buses 140). The transceiver module 130 may communicate bi-directionally, via the one or more antennas 135, wired links, and/or wireless links, with one or more networks or remote devices. For example, the transceiver module 130 may communicate bi-directionally with one or more of device 170-a, device 170-b, remote control device 155, and/or sensors 160-a, 160-d. The devices 170-a, 170-b may be components of the orthosis 1, or other devices in communication with the orthosis 1. The transceiver module 130 may include a modem to modulate the packets and provide the modulated packets to the one or more antennas 135 for transmission, and to demodulate packets received from the one or more antennas 135. In some embodiments (not shown) the transceiver may be communicate bi-directionally with one or more of device 170-a, device 170-b, remote control device 155, and/or sensors 160-a, 160-d through a hardwired connection without necessarily using antenna 135. While a control panel or a control device (e.g., 150) may include a single antenna 135, the control panel or the control device may also have multiple antennas 135 capable of concurrently transmitting or receiving multiple wired and/or wireless transmissions. In some embodiments, one element of control device 150 (e.g., one or more antennas 135, transceiver module 130, etc.) may provide a connection using wireless techniques, including digital cellular telephone connection, Cellular Digital Packet Data (CDPD) connection, digital satellite data connection, and/or another connection.

The signals associated with system 100 may include wireless communication signals such as radio frequency, electromagnetics, local area network (LAN), wide area network (WAN), virtual private network (VPN), wireless network (using 802.11, for example), 345 MHz, Z-WAVE®, cellular network (using 3G and/or LTE, for example), and/or other signals. The one or more antennas 135 and/or transceiver module 130 may include or be related to, but are not limited to, WWAN (GSM, CDMA, and WCDMA), WLAN (including BLUETOOTH® and Wi-Fi), WMAN (WiMAX), antennas for mobile communications, antennas for Wireless Personal Area Network (WPAN) applications (including RFID and UWB). In some embodiments, each antenna 135 may receive signals or information specific and/or exclusive to itself. In other embodiments, each antenna 135 may receive signals or information not specific or exclusive to itself.

In some embodiments, one or more sensor units 160 (e.g., angle, velocity, acceleration, force, temperature, etc.) may connect to some element of system 100 via a network using one or more wired and/or wireless connections. In some embodiments, the user interface module 125 may include an audio device, such as an external speaker system, an external display device such as a display screen, and/or an input device (e.g., remote control device interfaced with the user interface module 125 directly and/or through I/O controller module 120).

One or more buses 140 may allow data communication between one or more elements of control device 150 (e.g., processor module 105, memory 110, I/O controller module 120, user interface module 125, etc.).

The memory 110 may include random access memory (RAM), read only memory (ROM), flash RAM, and/or other types. The memory 110 may store computer-readable, computer-executable software/firmware code 115 including instructions that, when executed, cause the processor module 105 to perform various functions described in this disclosure (e.g., initiating an adjustment of a lighting system, etc.). Alternatively, the software/firmware code 115 may not be directly executable by the processor module 105 but may cause a computer (e.g., when compiled and executed) to perform functions described herein. Alternatively, the computer-readable, computer-executable software/firmware code 115 may not be directly executable by the processor module 105 but may be configured to cause a computer (e.g., when compiled and executed) to perform functions described herein. The processor module 105 may include an intelligent hardware device, e.g., a central processing unit (CPU), a microcontroller, an application-specific integrated circuit (ASIC), etc.

In some embodiments, the memory 110 can contain, among other things, the Basic Input-Output system (BIOS) which may control basic hardware and/or software operation such as the interaction with peripheral components or devices. For example, the actuation module 145, and other modules and operational components of the control device 150 used to implement the present systems and methods may be stored within the system memory 110. Applications resident with system 100 are generally stored on and accessed via a non-transitory computer readable medium, such as a hard disk drive or other storage medium. Additionally, applications can be in the form of electronic signals modulated in accordance with the application and data communication technology when accessed via a network interface (e.g., transceiver module 130, one or more antennas 135, etc.).

Many other devices and/or subsystems may be connected to one or may be included as one or more elements of system 100. In some embodiments, all of the elements shown in FIG. 8 need not be present to practice the present systems and methods. The devices and subsystems can be interconnected in different ways from that shown in FIG. 8. In some embodiments, an aspect of some operation of a system, such as that shown in FIG. 8, may be readily known in the art and are not discussed in detail in this application. Code to implement the present disclosure can be stored in a non-transitory computer-readable medium such as one or more of system memory 110 or other memory. The operating system provided on I/O controller module 120 may be iOS®, ANDROID®, MS-DOS®, MS-WINDOWS®, OS/2®, UNIX®, LINUX®, or another known operating system.

The transceiver module 130 may include a modem configured to modulate the packets and provide the modulated packets to the antennas 135 for transmission and/or to demodulate packets received from the antennas 135. While the control panel or control device (e.g., 205) may include a single antenna 135, the control panel or control device (e.g., 150) may have multiple antennas 135 capable of concurrently transmitting and/or receiving multiple wireless transmissions.

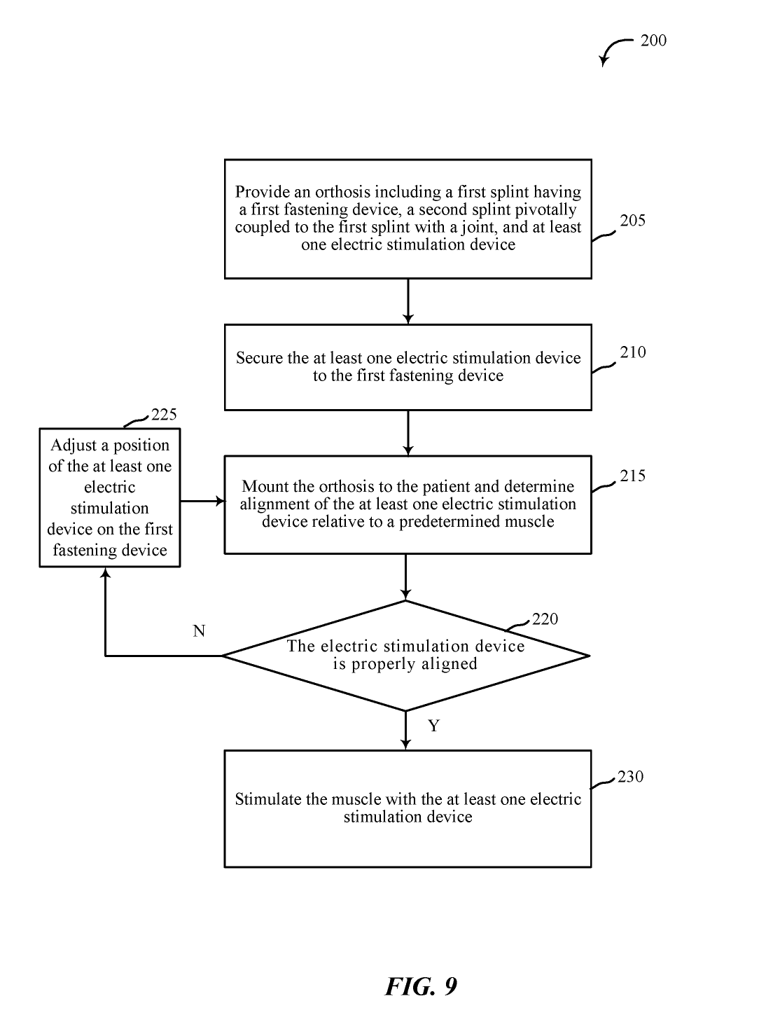

FIG. 9 is a flow chart illustrating an example of a method 200 for improving orthoses, in accordance with various aspects of the present disclosure. One or more aspects of the method 200 may be implemented in conjunction with orthosis 1 of FIGS. 1-7B, actuation module 145 depicted in FIG. 8, and/or control device 150 shown in FIG. 8. In some examples, a computing device may execute one or more sets of codes to control the functional elements of the computing device or aspects of the orthosis 1, actuation module 145, and/or control device 150 to perform one or more of the functions described below. Additionally or alternatively, the computing device may perform one or more of the functions described below using special-purpose hardware.

At block 205, method 200 may include providing an orthosis including a first splint having a first fastening device, a second splint pivotally coupled to the first splint with a joint, and at least one electric stimulation device. At block 210, the method 200 may include securing the at least one electric stimulation device to the first fastening device. Block 215 may include mounting the orthosis to the patient and determine alignment of the at least one electric stimulation device relative to a predetermined muscle. Block 220 may include determining wherein the electric stimulation device is properly aligned with the predetermined muscle. If the electric stimulation device is not properly aligned the method 200 may include adjusting a position of the at least one electric stimulation device on the first fastening device, followed by blocks 215 and 220. If the electric stimulation device is properly aligned with the muscle, the method 200 may also include, at block 230, stimulating the muscle with the at least one electric stimulation device.

In other embodiments, methods according to the present disclosure may include further steps in addition to those shown in FIG. 9. In some embodiments, the methods may include variations of the steps shown in FIG. 9, or few steps than those shown in FIG. 9. For example, the method 200 may further include adjusting a position of the first fastening device relative to the first splint in order to adjust a position of the at least one electric stimulation device relative to the muscle rather than, or in addition to, adjusting a position of the at least one electric stimulation device relative to the first fastening member. The method 200 may include mounting the at least one electric stimulation device directly to the first and/or second splint. The adjustability of the position of at least one electric stimulation device relative to other features of the orthosis may provide customized and repeatable contact of the at least one electric stimulation device relative to the intended muscle to be stimulated each time the orthosis is mounted to the patient.

The detailed description set forth above in connection with the appended drawings describes examples and does not represent the only instances that may be implemented or that are within the scope of the claims. The terms “example” and “exemplary,” when used in this description, mean “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and apparatuses are shown in block diagram form in order to avoid obscuring the concepts of the described examples.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and components described in connection with this disclosure may be implemented or performed with a general-purpose processor, a digital signal processor (DSP), an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, and/or state machine. A processor may also be implemented as a combination of computing devices, for example, a combination of a DSP and a microprocessor, multiple microprocessors, one or more microprocessors in conjunction with a DSP core, and/or any combination thereof.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items (for example, a list of items prefaced by a phrase such as “at least one of” or “one or more of”) indicates a disjunctive list such that, for example, a list of “at least one of A, B, or C” means A or B or C or AB or AC or BC or ABC, or A and B and C.

In addition, any disclosure of components contained within other components or separate from other components should be considered exemplary because multiple other architectures may potentially be implemented to achieve the same functionality, including incorporating all, most, and/or some elements as part of one or more unitary structures and/or separate structures.

Computer-readable media includes both computer storage media and communication media including any medium that facilitates transfer of a computer program from one place to another. A storage medium may be any available medium that can be accessed by a general purpose or special purpose computer. By way of example, and not limitation, computer-readable media can comprise RAM, ROM, EEPROM, flash memory, CD-ROM, DVD, or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, or any combination thereof, then the coaxial cable, fiber optic cable, twisted pair, DSL, or wireless technologies such as infrared, radio, and/or microwave are included in the definition of medium. Disk and disc, as used herein, include any combination of compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed.

The process parameters, actions, and steps described and/or illustrated in this disclosure are given by way of example only and can be varied as desired. For example, while the steps illustrated and/or described may be shown or discussed in a particular order, these steps do not necessarily need to be performed in the order illustrated or discussed. The various exemplary methods described and/or illustrated here may also omit one or more of the steps described or illustrated here or include additional steps in addition to those disclosed.

Furthermore, while various embodiments have been described and/or illustrated here in the context of fully functional computing systems, one or more of these exemplary embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may permit and/or instruct a computing system to perform one or more of the exemplary embodiments disclosed here.

This description, for purposes of explanation, has been described with reference to specific embodiments. The illustrative discussions above, however, are not intended to be exhaustive or limit the present systems and methods to the precise forms discussed. Many modifications and variations are possible in view of the above teachings. The embodiments were chosen and described in order to explain the principles of the present systems and methods and their practical applications, to enable others skilled in the art to utilize the present systems, apparatus, and methods and various embodiments with various modifications as may be suited to the particular use contemplated.

We claim:

1. An orthosis comprising:
a first splint having a first fastening device configured to fix the first splint to a first body part;
a second splint configured to contact a second body part when the orthosis is donned on a wearer;
a joint connecting the first splint to the second splint;
at least one electric stimulation device fastened to the orthosis and operable to stimulate at least one muscle of the first body part or the second body part.

2. The orthosis according to claim 1, further comprising a third splint connected to one of the first and second splints by a second joint, the third splint configured to contact a third body part when the orthosis is donned on the wearer.

3. The orthosis according to claim 1, further comprising at least one second fastening device disposed on the second splint and configured to fix the second splint to the second body part.

4. The orthosis according to claim 1, wherein the at least one electric stimulation device is fixed to one of the first and second splints or to the first fastening device.

5. The orthosis according to claim 1, wherein the at least one electric stimulation device is repositionable on the first or second splint or the first fastening device.

6. The orthosis according to claim 1, wherein the at least one electric stimulation device includes at least one electrode arrangement.

7. The orthosis according to claim 1, wherein the first and second splints are freely pivotable about a pivot axis that is formed by the joint.

8. The orthosis according to claim 1, wherein the second splint is configured as a foot part that is configured to contact a bottom surface of the wear's foot and the first splint is configured as a lower leg splint.

9. The orthosis according to claim 8, wherein the joint is arranged level in height with a natural ankle joint.

10. The orthosis according to claim 2, wherein the third splint is configured as a thigh splint and the second joint is arranged level in height with a natural knee joint.

11. The orthosis according to claim 1, wherein the first and second splints guide at least one of the first and second body parts in a medial-lateral direction.

12. The orthosis according to claim 1, wherein the first and second splints are adjustable in a reversible manner.

13. The orthosis according to claim 1, wherein the at least one electric stimulation device includes markings or fastening elements, which are positioned on the first or second splint or on the first fastening device.

14. The orthosis according to claim 1, wherein the at least one of the first and second splints includes adjustable stops.

15. An orthosis comprising:
a first splint having a first fastening device configured to fix the first splint to a first body part;
a second splint configured to contact a second body part when the orthosis is donned on a wearer;
a joint connecting the first splint to the second splint;
a third splint connected to one of the first and second splints by the joint, the third splint configured to contact a third body part when the orthosis is donned on the wearer;
at least one electric stimulation device fastened to the orthosis.

16. The orthosis according to claim 15, further comprising at least one second fastening device disposed on the second splint and configured to fix the second splint to the second body part.

17. The orthosis according to claim 15, wherein the at least one electric stimulation device is fixed to one of the first and second splints or to the first fastening device.

18. The orthosis according to claim 15, wherein the at least one electric stimulation device is repositionable on the first or second splint or the first fastening device.

19. The orthosis according to claim 15, wherein the at least one electric stimulation device includes at least one electrode arrangement.

20. The orthosis according to claim 15, wherein the first and second splints are freely pivotable about a pivot axis that is formed by the joint.
